(19) United States
(12) Patent Application Publication
(10) Pub. No.: US 2005/0055050 A1
(43) Pub. Date: Mar. 10, 2005

(54) Intravascular occlusion device

(76) Inventor: Arthur A. Alfaro, Colts Neck, NJ (US)

Correspondence Address: DILWORTH & BARRESE, LLP, 333 EARLE OVINGTON BLVD., UNIONDALE, NY, 11553, US

(21) Appl. No.: 10/896,247

(22) Filed: Jul. 21, 2004

Related U.S. Application Data
(60) Provisional application No. 60/490,480, filed on Jul. 24, 2003.

(51) Int. Cl. A61B 17/08

(52) U.S. Cl. 606/213

(57) ABSTRACT

A device for repairing an anatomical defect such as a patent foramen ovale possesses spring-like characteristics enabling it to be stored within, and discharged from, the distal end of a catheter when the device is in the compressed state and to assume a defect-occluding configuration when the device is in the expanded, or noncompressed, state.

### CROSS REFERENCE TO RELATED APPLICATION

This application claims the benefit of U.S. provisional patent application Ser. No. 60/490,480, filed Jul. 24, 2003, the entire contents of which are incorporated by reference herein.

### BACKGROUND OF THE INVENTION

The present invention relates to a device for repairing an anatomical defect. In particular, the device of the present invention relates to the closure of a physical anomaly such as a vascular aperture or an aperture in a septum including patent foramen ovale, patent ductus arteriosus, atrial septal defect, or ventricular septal defect.

In various body tissues, septal defects may occur either congenitally or as a result of operative procedures. Such defects may include abnormal openings, for example, in the cardiovascular system including the heart. Procedures, developed to introduce devices for closing such abnormal openings, are generally referred to as embolization—the therapeutic introduction of a substance into a vessel in order to occlude it. A septum is generally defined as a thin wall of muscle or other tissue, which divides two or more chambers or other areas within the body. The term “septal defect” generally refers to a perforation or other hole passing through a septum. Ventricular septal defects, atrial septal defects and patent ductus arteriosus are the three most common congenital cardiac malformations. These defects have been surgically corrected for decades.

Initially, septal defects were corrected by open-heart surgery during which the surgeon would have to open the chest of a patient and bypass the heart temporarily, e.g., by means of a mechanical heart or a “heart-lung machine.” The surgeon would then physically cut into the heart and suture small defects closed. In the case of larger defects, a patch of a biologically compatible material would be sewn onto the septum to cover the defect. An atrial septal defect makes the heart muscles work considerably harder because of shunting of blood through the defect and, if left untreated, leads to high pulmonary arterial pressures, and this additional strain placed on the heart muscles can cause fatal heart failure.

In order to avoid the morbidity and mortality associated with open-heart surgery, a variety of catheter closure techniques have been attempted. In such techniques, an occluding device is delivered through a catheter. Once the closure device or occluder is positioned adjacent to the defect, it must be attached to the rest of the septum in a manner which permits it to effectively block the passage of blood through the defect.

One type of the occluder associated with the catheter closure techniques has an umbrella-type actuating mechanism. Typically, the latter includes a string connecting numerous arms to an anchor, which includes an internal wire skeleton and a central, shaped piece of rubber. The string attached to the arms is affixed to the central rubber element of the anchor. The anchor is placed on the opposite side of the septum from the umbrella and the length of the string limits movement of the occlusion device with respect to the septum.

The occluder of the type described above may have a few drawbacks. Firstly, it may be mechanically complex and require a great deal of remote manipulation for deployment, such as by applying tension to one or more cables in order to deploy the arms of an umbrella or to anchor the device in place. This extensive remote manipulation not only increases the difficulty of the procedure, but also tends to increase the likelihood that the device will be improperly deployed and require either retrieval or repositioning.

Secondly, the umbrella-type occluder has essentially two separate members, which are joined to each other at a single point or pivot. When the left member is opened, the central point would tend to ride to the lower margin of the defect; proper centering of the device, which is critical to a successful outcome, may be excessively challenging.

To avoid the above-discussed difficulties, mechanically operated occluders have been partially substituted with occluders made from shape-memory alloys. Such an occluder tends to assume the desired shape in response to a predetermined temperature. It has been observed that, sometimes, an occluder made from the shape-memory alloys tends to undergo certain changes while being guided through a catheter. The premature transformation of the shape of the occluder may complicate the delivery thereof and compromise its configuration. As a consequence, a septal defect may not be adequately closed, and the occluder either should be replaced or manipulated within the defect, which is highly undesirable for the health reasons.

Structurally, many types of occluders can be generally characterized as coil embolization devices. The coil-type occlusion devices may be associated with a number of drawbacks that could be significant in some applications. Intravascular stability of the coils has been shown to be highly dependent on proper matching of coil diameter with the diameter of the target vessel. Moreover, a long vascular segment is often obliterated because of the frequent need for multiple coils and the coils often remain elongated within the vessel because their unconstrained diameter is larger than the vascular lumen.

These and other drawbacks have inspired modifications in the design and technique of coil embolization. Recently, detachable microcoils and macrocoils with controlled delivery have been designed to achieve a more compact conglomerate of the coil and to prevent migration by allowing optimal positioning of the coil before release. However, since optimal arrangement of the coil alone may not prevent migration in some cases, such as high flow conditions or venous placement, a coil anchoring system has been devised. Although an anchoring system may stabilize a coil conglomerate within the vasculature, significantly reducing or eliminating the possibility of coil migration, such a system may render the coil non-repositionable.

The need therefore exists for an embolization device having an easily deployable structure that reliably occludes an abnormal anatomical opening.

### SUMMARY OF THE INVENTION

To meet this need, an occluder device configured according to the invention includes at least one uniwire capable of assuming the desired shape corresponding to the shape of the anatomical defect to be occluded after the device has been deployed within the opening.

In accordance with one aspect of the invention, an occlusion device includes a uniwire coil made from a non-woven material and characterized by the inherent springing ability of the material to assume the desired shape after the device has been deployed in the anatomical defect. The uniwire coil including numerous turns is configured so that the its opposite ends, juxtaposed with the opposite surfaces of the anatomical defect, tend to compress toward one another and reliably engage the juxtaposed surfaces. Concomitantly, the compression of the opposite ends of the occlusion device is accompanied by the radial expansion of the middle portion shaped and sized to urge against the peripheral wall of the anatomical defect. Accordingly, the deployment of the occlusion device results in its reliable positioning, occluding and centering within the anatomical defect.

In accordance with another aspect of the invention, the uniwire coil may have only one free end carrying a retrieval nub made from a magnetic material. Accordingly, the localization of the nub, which attracts a positioning or retrieval device configured to reposition or remove the occluder from situ, is greatly facilitated.

It is therefore an object of the invention to provide a simple occluder deliverable through a small size catheter.

Yet another object of the invention is to provide an occluder easily deployable in situ.

A further object of the invention is to provide an occluder reliably occluding the defect.

### BRIEF DESCRIPTION OF THE DRAWINGS

The above and other features, objects and advantages will be come more readily apparent from the following descriptions illustrated by the drawings, in which:

FIGS. 1A-1E illustrate an occluder, configured in accordance with one embodiment of the invention, and a sequence of deployment of the inventive occluder in situ;

FIGS. 2A-2C illustrate a further embodiment of the inventive occluder;

FIGS. 3A-3B show top and bottom views of the inventive occluder of FIGS. 1 and 2;

FIGS. 4A-4E illustrate modification of the central portion of the inventive occluder;

FIGS. 6A-6E illustrate various embodiments of the inventive occluder; and

FIGS. 7A-7E illustrate different modifications of a retrieval device.

### DETAILED DESCRIPTION OF THE PREFERRED EMBODIMENT

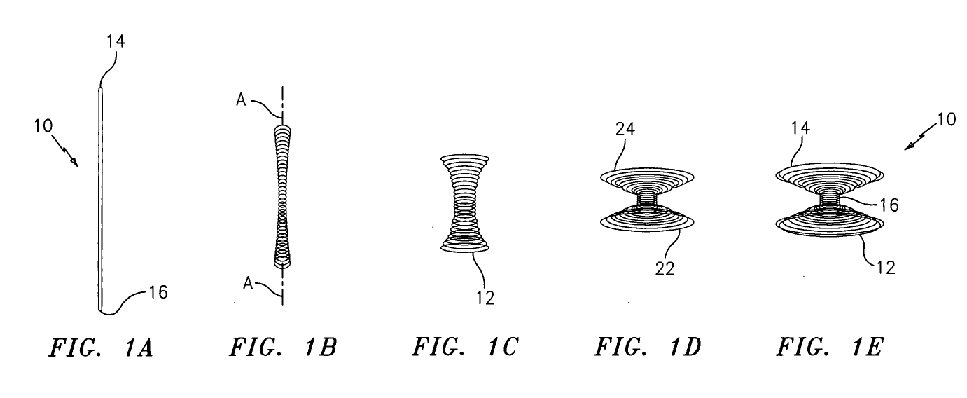

FIGS. 1-5 illustrate an occluder having a body 10 and a method of its deployment in occluding a patent foramen ovale (PFO). However, the device of this invention can also be successfully applied to occluding numerous septal defects including, but not limited to VSD, ASD and PDA. Device 10 includes a uniwire 12 made from a non-woven material that may include, but not be limited to, platinum, gold, rhodium, rhenium, palladium, tungsten, and alloys thereof. While the materials used for manufacturing device 10 are not made from shape-memory alloys, each of them has a springing characteristic allowing the device to transform its shape under certain conditions.

Capitalizing on the spring-like characteristics of the material used for making device 10, the device can be collapsed into its compressed state assuming an essentially linear configuration (FIG. 1) and in this state inserted into the lumen of a catheter at the distal (discharge) end of the latter (not shown). For example, the device of this invention when compressed may have an elongate configuration in its longitudinal axis A-A (see FIGS. 1A and 1B). The compressed configuration shown can be achieved simply by stretching device 10 generally along its axis, e.g. by manually grasping its opposite ends 14 and 16, and pulling them apart. Loading the compressed device into a catheter may be done at the time of implantation or, preferably, in advance of implantation. As device 10 is released from the catheter, it will tend to gradually and resiliently return to a preferred relaxed, i.e., noncompressed, shape going through the various stages illustrated in FIGS. 1B-1E. When the device springs back into the relaxed shape, it tends to act against the distal end of the catheter effectively urging itself forward beyond the end of the catheter. The operator can ensure the proper positioning of the device during its deployment by controlling the spring-like action of the opposite ends of device 10.

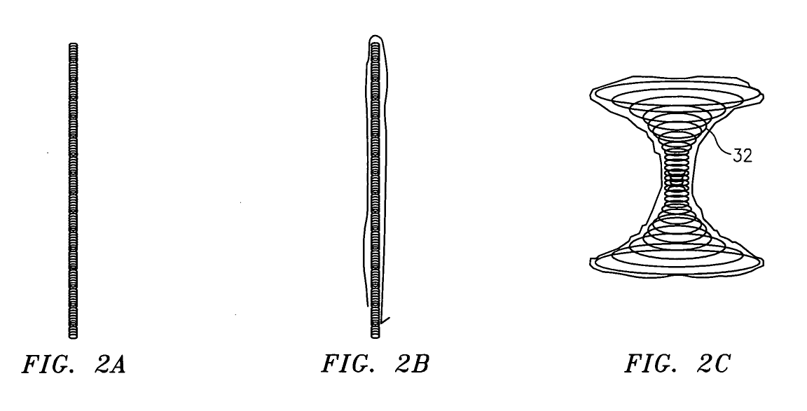

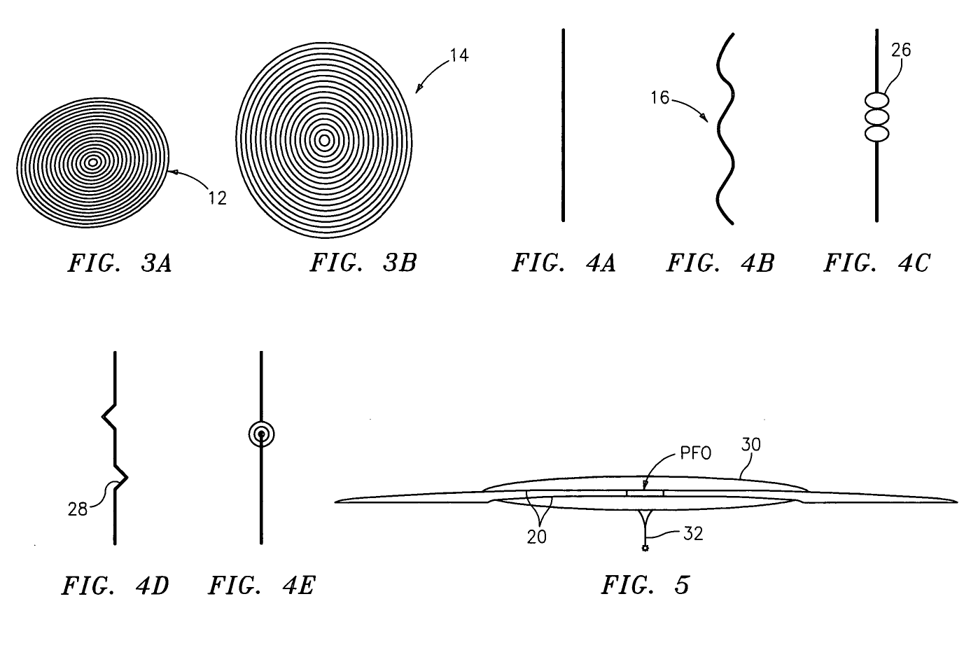

In its relaxed, or rest, state (see FIG. 2), device 10 generally includes two aligned panels 12 and 14 which can assume a variety of configurations, including that of discs (FIGS. 3A and 3B), linked together by a resilient central portion 16. The inner surface 20 of each disc (FIG. 5) may be concave, or cupped, to ensure that each of discs 12 and 14 contacts the septal wall. In one embodiment, loops 32 (FIG. 2C) form a substantially conical coil having a constant pitch. Alternatively, loops 32 can form a substantially conical coil having a variable pitch.

When device 10 is in its relaxed state, opposite discs 12 and 14 define therebetween a central portion 16. Advantageously, the latter does not provide a spring-like action (see FIG. 4A) which is generated only by the opposite ends causing perimeter edges 22 and 24 (FIG. 1D), respectively, to fully engage the sidewall of the septum (see FIG. 5).

However, central portion 16 can be formed to provide an additional spring-like action. As illustrated in FIGS. 4B-4E, central portion 16 may have a variety of shapes configured to compress after the device 10 has been deployed in vivo so as to bring opposite ends 12 and 14 of the device toward each other. Whether central portion 16 has a sinusoidal shape (FIG. 4B), at least one annular loop 26 (FIG. 4C), at least one trough-like formation 28 (FIG. 4D or a plurality of concentric circles (FIG. 4E), it is flexible in both the lateral and fore and aft directions. This flexibility provides for the self-centering of the device, wherein discs 12 and 14 tend to automatically center themselves around the adjacent opening of the defect while tending to pull the discs toward each other.

Those skilled in the art will appreciate that device 10 will be sized in proportion to the opening to be occluded. The diameter of each end 12 and 14 may be varied as desired for differently sized openings in the septal wall. Further, the length of resilient central portion 16 may be varied depending upon the thickness of the septal wall.

To minimize areas of statis and to improve anchoring of device 10, the latter may be encapsulated within a non-absorbable or absorbable material including, for example, dacron, nylon, polypropylene, gelatin, polyglactin, and the like. A mesh member 30 (FIG. 5) made from one of afore-listed materials or other suitable material is permeable to allow for tissue ingrowth. In one embodiment, mesh member 30 comprises a biocompatible material connected to the uniwire body 10. Alternatively, mesh member 30 may comprise a variety of suitable permeable structures which support epithelialization, as for example, where the mesh member comprises walls connected together to form a sock (not shown). As shown in FIGS. 2A-2C, mesh member 30 extends along the entire length of device 10 from first end 12 to second end 14 thereof. In another embodiment, mesh member 30 is selectively attached to the occluder 10 at spaced-apart locations, preferably, covering the tops of the ends 12 and 14 facing away from the tissue. Attachment of mesh member 30 to device 10 may include adhesive, heat bonding, solvent bonding, or the like.

Preferably, disc-shaped ends 12 and 14 of device 10 each have a slight concavity of inner surface 20 (FIG. 5) in the deployed state of the device. It has been found that as concave ends 12 and 14 urge against the tissue, thrombus formation and areas of statis are minimized. To reposition or recapture device 10 after its deployment, a magnetic retrieval nub 32 is advantageously provided on its proximal end.

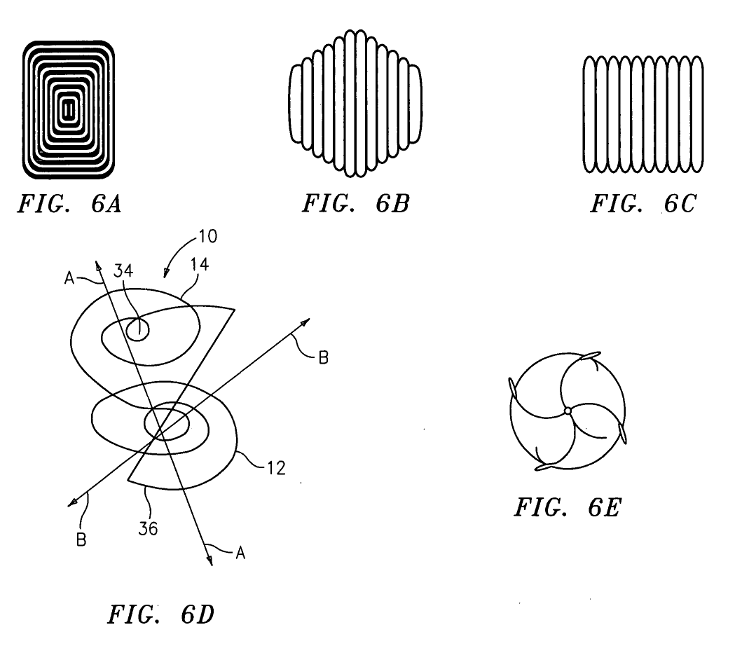

Alternative embodiments of device 10 made from unwoven material are illustrated in FIGS. 6A-6E. Instead of disc-shaped ends, the ends of device 10 may have a polygonal shape as seen in FIG. 6A. Conforming to the shape of the defect, the device may have a generally oval shape as illustrated in FIG. 6B or a cylindrical shape as illustrated in FIG. 6C. Turning to FIG. 6D, device 10 includes an endless, uniwire structure configured to have its opposite ends centered about axes B-B and C-C, which extend transversely to one another in the semi-deployed state of the device. Opposite wire ends 34 and 36 are interconnected by central portion 16 configured to compress opposite ends 12 and 14 to a fully deployed state wherein these ends each cover a respective side of the defect to be occluded. FIG. 6E illustrates only one of the ends of device 10 provided with a flower pedal design which includes multiple loops encircled by a peripheral collar.

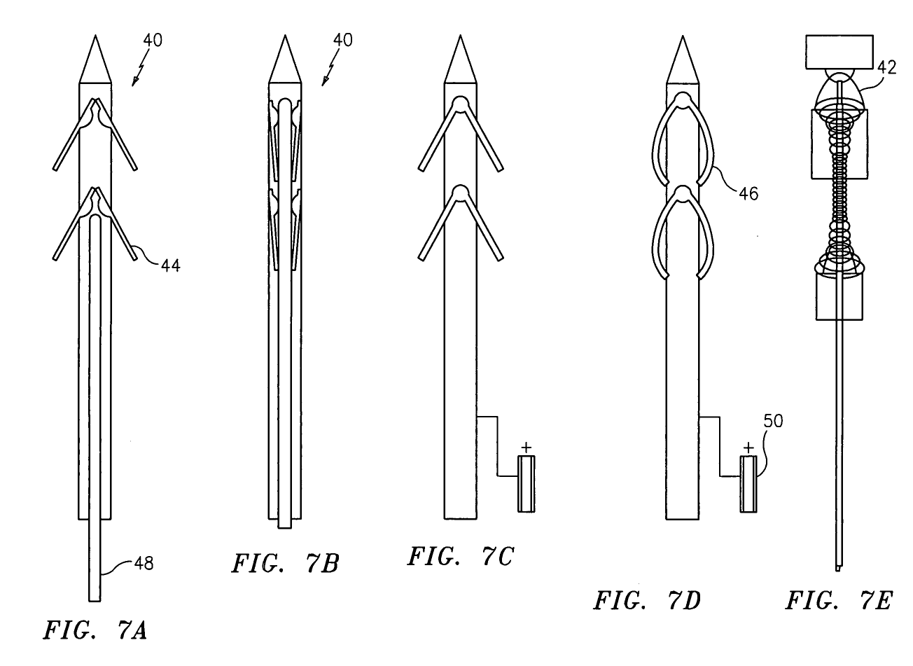

Device 10 may be retrieved from, or repositioned, by means of mechanical or even electrically actuated retrieval devices as illustrated in FIG. 7. As mentioned before, device 10 may be provided with magnetic nub 32 that can be advantageously used to automatically attract the magnetizable tip of the retrieval device. Alternatively, either both or one of ends 12 and 14 may be provided with a handle 42 (FIG. 7E) engageable by mechanically operated arms 44 (FIGS. 7A-7B and FIGS. 7C-7D, respectively). Utilization of a mechanical actuator can be provided by a simple push rod 48, the linear motion of which translates into the pivoting motion of arms 44. Alternatively, as illustrated in FIGS. 7C-7D, an electrical battery or otherwise operated actuator 50 can provide the displacement of the arms from a rest position to an expanded position in which arms 44 engage handle 42.

Various modifications and improvements may be made to the present invention without departing from the scope of the features as enumerated hereinbelow.

1. A device for occluding an anatomical defect comprising a coil exhibiting spring-like characteristics and made from a material other than a shape-memory alloy, the coil in its compressed state possessing an essentially linear configuration adapted to be accommodated by, and discharged from, the distal end of a catheter and in its non-compressed, or relaxed, state, possessing opposite ends interconnected along their common axis by a central portion, the opposite ends extending radially outward from their common axis to form panels disposed approximately transversely to said axis, the panels tending to be urged toward each other due to their spring-like characteristics.

2. The device of claim 1 in which the coil is fabricated from an endless uniwire.

3. The device of claim 2 wherein the endless uniwire is made of metal or metal alloy.

4. The device of claim 3 wherein the metal alloy is fabricated from one or more of platinum, gold, rhodium, rhenium, palladium, tungsten or alloy thereof.

5. The device of claim 1 wherein the inner surfaces of the panels assume a concave configuration.

6. The device of claim 1 wherein the panels form conical coils possessing a constant or variable pitch.

7. The device of claim 1 wherein the central portion of the coil in its non-compressed, or relaxed, state exhibits little or no spring-like action.

8. The device of claim 1 wherein the central portion of the coil in its non-compressed, or relaxed, state exhibits a spring-like action.

9. The device of claim 1 wherein the central portion of the coil possesses a sinusoidal configuration.

10. The device of claim 1 wherein the central portion of the coil possesses a least one annular loop.

11. The device of claim 1 wherein the central portion of the coil possesses a trough-like configuration.

12. The device of claim 1 wherein the central portion of the coil possesses a plurality of concentric circles.

13. The device of claim 1 encapsulated within a permeable structure allowing for tissue ingrowth.

14. The device of claim 1 possessing means for facilitating its retrieval from, or repositioning within, a body in which it has been deployed.

15. The device of claim 14 wherein said means is a magnetic nub or a handle engageable by a device-retrieving or repositioning instrument.

16. A method for occluding an opening in an anatomical structure which comprises deploying the device of claim 1.

17. The method of claim 16 wherein the device is deployed by inserting the device in its compressed state within the distal end of a catheter; positioning the distal end of the catheter beyond the far side of the opening in the anatomical structure; partially discharging the device from the distal end of the catheter whereupon the discharged position of the device assumes its non-compressed, or relaxed, state on the far side of the opening; repositioning the distal end of the catheter to the near side of the opening in the anatomical structure; and, completing the discharging of the device from the distal end of the catheter whereupon the remaining discharged portion of the device assumes its non-compressed, or relaxed, state on the near side of the opening, the device in its now fully deployed configuration occluding the opening in the anatomical structure.

18. The method of claim 16 wherein the opening to be occluded is a vascular aperture or an aperture in a septum.

19. The method of claim 16 wherein the opening is a patent foramen ovale, patent ductus arteriosus, atrial septal defect or ventricular septal defect.

20. The method of claim 17 wherein the opening is a patent foramen ovale, patent ductus arteriosus, atrial septal defect or ventricular septal defect.
